SEQUENCE LISTING (38 sequences)

SEQ ID NO: 1 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 21 aa, Homo sapiens
GIVEQCCTSICSLYQLENYCN

SEQ ID NO: 3 - protein, 30 aa, Homo sapiens
FVNQHLCGSHLVEALYLVCGERGFFYTPKT

SEQ ID NO: 4 - protein, 64 aa, Artificial
  Synthetic Insulin Analogue
FVNQHLCGSHLVEALYLVCGERGFFYTPKTXXXXXXXXXXXXXGIVEQCCTSICSLYQLENYCN

SEQ ID NO: 5 - protein, 61 aa, Artificial
  Synthetic Insulin Analogue
FVNQHLCGSHLVEALYLVCGERGFFYTPKTRXXXXXXXXRGIVEQCCTSICSLYQLENYCN

SEQ ID NO: 6 - protein, 61 aa, Artificial
  Synthetic Insulin Analogue
FVNQHLCGSHLVEALYLVCGERGFFYTPKTRRXXXXXXKRGIVEQCCTSICSLYQLENYCN

SEQ ID NO: 7 - protein, 61 aa, Artificial
  Synthetic Insulin Analogue
FVNQHLCGSHLVEALYLVCGERGFFYTPKTRREXXXXQKRGIVEQCCTSICSLYQLENYCN

SEQ ID NO: 8 - protein, 57 aa, Artificial
  Synthetic Insulin Analogue
FVNQHLCGSHLVEALYLVCGERGFFYTPKTRREQKRGIVEQCCTSICSLYQLENYCN

SEQ ID NO: 9 - protein, 58 aa, Artificial
  Synthetic Insulin Analogue
FVNQHLCGSHLVEALYLVCGERGFFYTPKTRREAQKRGIVEQCCTSICSLYQLENYCN

SEQ ID NO: 10 - protein, 58 aa, Artificial
  Synthetic Insulin Analogue
FVNQHLCGSHLVEALYLVCGERGFFYTPKTRRELQKRGIVEQCCTSICSLYQLENYCN

SEQ ID NO: 11 - protein, 59 aa, Artificial
  Synthetic Insulin Analogue
FVNQHLCGSHLVEALYLVCGERGFFYTPKTRREALQKRGIVEQCCTSICSLYQLENYCN

SEQ ID NO: 12 - protein, 60 aa, Artificial
  Synthetic Insulin Analogue
FVNQHLCGSHLVEALYLVCGERGFFYTPKTRREAELQKRGIVEQCCTSICSLYQLENYCN

SEQ ID NO: 13 - protein, 60 aa, Artificial
  Synthetic Insulin Analogue
FVNQHLCGSHLVEALYLVCGERGFFYTPKTRREASLQKRGIVEQCCTSICSLYQLENYCN

SEQ ID NO: 14 - protein, 61 aa, Artificial
  Synthetic Insulin Analogue
FVNQHLCGSHLVEALYLVCGERGFFYTPKTRREAESLQKRGIVEQCCTSICSLYQLENYCN

SEQ ID NO: 15 - protein, 61 aa, Artificial
  Synthetic Insulin Analogue
FVNQHLCGSHLVEALYLVCGERGFFYTKPTXXXXXXXXXXGIVEQCCTSICSLYQLENYCN

SEQ ID NO: 16 - protein, 61 aa, Artificial
  Synthetic Insulin Analogue
FVNQHLCGSHLVEALYLVCGERGFFYTPDTXXXXXXXXXXGIVEQCCTSICSLYQLENYCN

SEQ ID NO: 17 - protein, 61 aa, Artificial
  Synthetic Insulin Analogue
FVNQHLCGSDLVEALYLVCGERGFFYTPKTXXXXXXXXXXGIVEQCCTSICSLYQLENYCN

SEQ ID NO: 18 - protein, 61 aa, Artificial
  Synthetic Insulin Analogue
FVNQHLCGSDLVEALYLVCGERGFFYTPKTRREXXXXQKRGIVEQCCTSICSLYQLENYCN

SEQ ID NO: 19 - protein, 6 aa, Artificial
  Synthesized Construct
GGGPRR

SEQ ID NO: 20 - protein, 5 aa, Artificial
  Synthesized Construct
GGPRR

SEQ ID NO: 21 - protein, 6 aa, Artificial
  Synthesized Construct
GSEQRR

SEQ ID NO: 22 - protein, 6 aa, Artificial
  Synthesized Construct
RREQKR

SEQ ID NO: 23 - protein, 8 aa, Artificial
  Synthesized Construct
RREALQKR

SEQ ID NO: 24 - protein, 6 aa, Artificial
  Synthesized Construct
GAGPRR

SEQ ID NO: 25 - protein, 4 aa, Artificial
  Synthesized Construct
GPRR

SEQ ID NO: 26 - protein, 57 aa, Artificial
  Single Chain Insulin Analogue
FVNQHLCGSHLVEALYLVCGERGFFYTDPTGGGPRRGIVEQCCHSICSLYQLENYCN

SEQ ID NO: 27 - DNA, 171 bp, Homo Sapiens
ttcgtcaaccagcacctctgcggcagcgacctcgtcgaagcactctacctcgtctgcggagaacgaggattcttctacacagacccaacaggaggaggaccacgacgaggaatagtagaacaatgctgccacagcatatgtagcctctaccaactagaaaactactgcaac

SEQ ID NO: 28 - DNA, 171 bp, Homo Sapiens
ttcgtcaaccagcacctctgcggcagccacctcgtcgaagcactctacctcgtctgcggagaacgaggattcttctacacagacccaacaggaggaggaccacgacgaggaatagtagaacaatgctgccacagcatatgtagcctctaccaactagaaaactactgcaac

SEQ ID NO: 29 - DNA, 171 bp, Homo Sapiens
tttgttaaccaacatttgtgtggttctgatttggttgaagctttgtacttggtttgtggtgaaagaggttttttttacactgatccaactggtggtggtccaagaagaggtattgttgaacaatgttgtcattctatttgttctttgtaccaattggaaaactactgtaac

SEQ ID NO: 30 - protein, 30 aa, Artificial
  Synthetic Insulin B-chain Analogue
FVNQHLCGSHLVEALYLVCGERGFFYTKPT

SEQ ID NO: 31 - protein, 21 aa, Artificial
  Synthetic Insulin A-chain Analogue
GIVEQCCTSICSWYQLENYCN

SEQ ID NO: 32 - protein, 21 aa, Artificial
  Synthetic Insulin A-chain Analogue
GIVEQCCTSICSLWQLENYCN

SEQ ID NO: 33 - protein, 58 aa, Artificial
  Synthetic Single Chain Insulin Analogue
FVNQHLCGSHLVEALYLVCGERGFFYTPKTGGGPGKRGIVEQCCTSICSLYQLENYCN

SEQ ID NO: 34 - protein, 30 aa, Artificial
  Human insulin B-chain peptide analogue
FVNQHLCGSDLVEALYLVCGERGFFYTDPT

SEQ ID NO: 35 - protein, 21 aa, Artificial
  Human insulin A-chain peptide analogue
GIVEQCCHSICSLYQLENYCN

SEQ ID NO: 36 - protein, 57 aa, Artificial
  Synthetic single chain insulin analogue
FVNQHLCGSDLVEALYLVCGERGFFYTDPTGGGPRRGIVEQCCHSICSLYQLENYCN

SEQ ID NO: 37 - protein, 21 aa, Artificial
  Human insulin A-chain or analogue thereof
GIVEQCCXSICSLYQLENYCN

SEQ ID NO: 38 - protein, 30 aa, Artificial
  Human insulin B-chain or analogue thereof
FVNQHLCGSXLVEALYLVCGERGFFYTXXT

(19) United States
(12) Patent Application Publication
(10) Pub. No.: US 2011/0195896 A1
(43) Pub. Date: Aug. 11, 2011

(54) ISOFORM-SPECIFIC INSULIN ANALOGUES

(75) Inventors: Michael Weiss, Moreland Hills, OH (US); Jonathan Whittaker, Shaker Heights, OH (US)

(73) Assignee: CASE WESTERN RESERVE UNIVERSITY, Cleveland, OH (US)

(21) Appl. No.: 12/989,399

(22) PCT Filed: Apr. 22, 2009
(86) PCT No.: PCT/US2009/041439

§ 371 Date: Jan. 21, 2011

Related U.S. Application Data
(60) Provisional application No. 61/046,985, filed on Apr. 22, 2008.

(51) Int. Cl. A61K 38/28; C07K 14/62; C12N 15/17; C12N 15/63; C12N 5/00; A61P 3/10

(52) U.S. Cl. 514/5.9; 530/303; 536/23.51; 435/320.1; 435/325

(57) ABSTRACT

A method treating a mammal by administering a physiologically effective amount of an insulin analogue or a physiologically acceptable salt thereof where the insulin analogue displays more than twofold greater binding affinity to insulin receptor isoform A (IR-A) than insulin receptor isoform B (IR-B). The insulin analogue may be a single-chain insulin analogue or a physiologically acceptable salt thereof, containing an insulin A-chain sequence or an analogue thereof and an insulin B-chain sequence or an analogue thereof connected by a polypeptide of 4-13 amino acids. A single-chain insulin analogue may display greater in vitro insulin receptor binding to IR-A but lower binding to IR-B than normal insulin while displaying less than or equal binding to IGFR than normal insulin.

### STATEMENT REGARDING FEDERALLY SUPPORTED RESEARCH OR DEVELOPMENT

This invention was made with government support under cooperative agreements awarded by the National Institutes of Health, Contract Nos. NIH R01DK069764, R01-DK74176, and R01-DK065890. The U.S. government may have certain rights to the invention.

### BACKGROUND OF THE INVENTION

Administration of insulin has long been established as a treatment for diabetes mellitus. Insulin is the product of a single-chain precursor, proinsulin, in which a connecting region (35 residues) links the C-terminal residue of B chain (residue B30) to the N-terminal residue of the A chain (FIG. 1A). Although the structure of proinsulin has not been determined, a variety of evidence indicates that it consists of an insulin-like core and disordered connecting peptide (FIG. 1B). Formation of three specific disulfide bridges (A6-A11, A7-B7, and A20-B19; FIG. 1B) is thought to be coupled to oxidative folding of proinsulin in the rough endoplasmic reticulum (ER). Proinsulin assembles to form soluble Zn²⁺-coordinated hexamers shortly after export from ER to the Golgi apparatus. Endoproteolytic digestion and conversion to insulin occurs in immature secretory granules followed by morphological condensation. Crystalline arrays of zinc insulin hexamers within mature storage granules have been visualized by electron microscopy (EM). Assembly and disassembly of native oligomers is thus intrinsic to the pathway of insulin biosynthesis, storage, secretion, and action.

Amino-acid substitutions in the A- and/or B chains of insulin have widely been investigated for possible favorable effects on the pharmacokinetics of insulin action following subcutaneous injection. Examples are known in the art of substitutions that accelerate or delay the time course of absorption. Such substitutions (such as Asp^{B28} in Novalog® and [Lys^{B28}, Pro^{B29}] in Humalog®) can be and often are associated with more rapid fibrillation and poorer physical stability. Indeed, a series of ten analogues of human insulin for susceptibility to fibrillation, including Asp^{B28}-insulin and Asp^{B10}-insulin have been tested. All ten were found to be more susceptible to fibrillation at pH 7.4 and 37° C. than is human insulin. The ten substitutions were located at diverse sites in the insulin molecule and are likely to be associated with a wide variation of changes in classical thermodynamic stability. These results suggest that substitutions that protect an insulin analogue from fibrillation under pharmaceutical conditions are rare; no structural criteria or rules are apparent for their design. The present theory of protein fibrillation posits that the mechanism of fibrillation proceeds via a partially folded intermediate state, which in turn aggregates to form an amyloidogenic nucleus. In this theory, it is possible that amino-acid substitutions that stabilize the native state may or may not stabilize the partially folded intermediate state and may or may not increase (or decrease) the free-energy barrier between the native state and the intermediate state. Therefore, the current theory indicates that the tendency of a given amino-acid substitution in the insulin molecule to increase or decrease the risk of fibrillation is highly unpredictable.

Modifications of proteins such as insulin are known to increase resistance to fibrillation but impair biological activity. For example, “mini-proinsulin,” is used to describe a variety of proinsulin analogues containing shortened linker regions such as a dipeptide linker between the A and B chains of insulin. Additional substitutions may also be present such as Ala^{B30} found in porcine insulin instead of Thr^{B30} as found in human insulin. This analogue is sometimes referred to as Porcine Insulin Precursor, or PIP. Mini-proinsulin analogues are frequently resistant to fibrillation but are impaired in their activity. In general, connecting peptides of length <4 residues block insulin fibrillation at the expense of biological activity; affinities for the insulin receptor are reported to be reduced by at least 10,000-fold. While such analogues are useful as intermediates in the manufacture of recombinant insulin, they are not useful per se in the treatment of diabetes mellitus.

Insulin mediates its biological actions by binding to and activating a cellular receptor, designated the insulin receptor. The extracellular portion of the insulin receptor binds insulin whereas the intracellular portion contains a hormone-activatable tyrosine-kinase domain. Alternative RNA splicing leads to two distinct isoforms of the insulin receptor (IR), designated IR-A and IR-B. The β isoform contains twelve additional amino acids in the α-subunit, encoded by exon 11 of the insulin receptor gene. The A isoform lacks this twelve-residue segment. The present invention concerns the design of insulin analogues that bind preferentially to one isoform of the insulin receptor.

Insulin analogues with affinities too low or too high for the insulin receptor may have unfavorable biological properties in the treatment of diabetes mellitus. Because clearance of insulin from the bloodstream is mediated primarily by interactions with the insulin receptor on target tissues, receptor-binding activities less than 25% would be expected to exhibit prolonged lifetimes in the bloodstream. Such delayed clearance would be undesirable in a fast-acting insulin analogue administered in coordination with food intake for the tight control of glycemia. Such reduced affinities would also decrease the potency of the insulin analogue, requiring injection of either a larger volume of protein solution or use of a more highly concentrated protein solution. The present invention concerns the design of insulin analogues that bind preferentially to one isoform of the insulin receptor.

Conversely, insulin analogues with affinities for the insulin receptor higher than that of wild-type insulin may be associated with altered signaling properties and altered cellular processing of the hormone-receptor complex. A prolonged residence time of the complex between the super-active insulin analogue and the insulin receptor on the surface of a target cell or on the surface of an intracellular vescicle may lead to elevated mitogenic signaling. Enhanced mitiogenicity can occur if the amino-acid substitutions not only augment binding of the analogue to the insulin receptor, but also to the Type I IGF receptor. For these reasons, it is desirable to have analogues whose affinities for the insulin receptor and IGF receptor are similar to those of wild-type human insulin.

A modification of insulin (substitution of His^{B10} by Asp) has been described that enhances the thermodynamic stability of insulin and also augments its affinity for the insulin receptor by twofold. Because this substitution blocks the binding of zinc and prevents the assembly of insulin dimers into hexamers, it was investigated as a candidate fast-acting analog. Clinical development was stopped, however, when Asp^{B10}-insulin was found to exhibit increased mitogenicity, increased cross-binding to the insulin receptor, and elevated rates of mammary tumor formation on chronic administration to Sprague-Dawley rats. Because the otherwise favorable properties of Asp^{B10}-insulin and possibly other insulin analogues are confounded by these adverse properties, it would be desirable to have a design method to retain the favorable properties conferred by such substitutions while at the same time avoiding the adverse properties. A particular example would be re-design of the insulin molecule to retain the enhanced thermodynamic stability and receptor-binding properties associated with substitution of His^{B10} by Asp without incurring increased cross-binding to the Type I IGF receptor or increased mitogenicity.

Although a primary function of insulin is to regulate the concentration of glucose in the blood, the hormone regulates multiple target tissues and physiological responses. Classical target tissues are muscle, fat and liver. Non-classical targets of insulin include the pancreatic β-cell, neurons of the central nervous system involved in the control of appetite, satiety and body weight, neurons of the peripheral nervous system, and white blood cells involved in inflammation and host defense. Each of these tissues exhibits a specific pattern of expression of IR-A and IR-B. Evidence suggests that signaling through IR-A and IR-B can activate different post-receptor pathways leading to differential effects on insulin-regulated glucose uptake, on the expression of insulin-regulated genes, and on cell growth and proliferation.

To date, there are no insulin analogues that distinguish between IR-A and IR-B with sufficient specificity to enable the selective activation of one signaling pathway or the other. Wild-type insulin binds with slightly higher affinity to IR-A than to IR-B (between one- and twofold binding preference for IR-A). Such analogues seemed unlikely to exist as the two receptor isoforms share the major domains responsible for hormone binding. Because the protein sequences present in IR-B but absent in IR-A contain only 12 amino-acid residues and because these residues are extrinsic to shared sites of hormone binding, it seemed likely that amino-acid substitutions that augmented or impaired the binding of an insulin analogue to IR-A would equally modulate the binding of that insulin analogue to IR-B. Our studies of conventional insulin analogues (see below) are consistent with this expectation.

Unexpectedly, we have discovered that a non-conventional class of insulin analogues, those containing a foreshortened connecting peptide between the A- and B-chains with modified A- and B-chains, can be designed to bind preferentially to IR-A. The overall organization of such analogues is analogous to proinsulin, the single-chain precursor of insulin in the biosynthetic pathway of hormone synthesis in the pancreatic β-cell. Human proinsulin contains a connecting region that links the C-terminal residue of the B-chain (residue B30) to the N-terminal residue of the A-chain (FIGS. 1A & B), and any isoform-specific effects of foreshortening this connecting domain are not known in the art.

An example of an insulin analogue that binds with greater affinity to IR-A than to IR-B is wild-type human proinsulin. Although fourfold selectivity in receptor binding is observed, in each case such binding is markedly impaired by the connecting domain, precluding its utility. Another example of an insulin-like ligand that binds with greater affinity to IR-A than to IR-B is insulin-like growth factor II (IGF-II). Like proinsulin, the extent of selectivity is between fourfold and tenfold. Use of IGF-II as an insulin analogue for the purposes of either laboratory investigation or treatment of humans with diabetes mellitus is undesirable because IGF-II binds with high activity to and activates the Type I IGF receptor (IGFR) whereas IGF-II has low affinity for either IR isoform (<20% relative to human insulin). Cross-binding of insulin analogues to IGFR has been associated with the development of mammary tumors in Sprague-Dawley rats. Use of IGF-II as a potential treatment for diabetes mellitus is also complicated by its binding to specific serum binding proteins, which alter the potency and signaling properties of this growth factor.

The marked sequence differences between proinsulin and IGF-II render it unclear how to design novel analogues that might exhibit the following combination of properties: (a) greater isoform selectivity than these naturally occurring ligands while at the same time exhibiting (b) an affinity for the targeted isoform equal to or greater than that of wild-type insulin and (c) cross-binding to IGFR similar to or lower than that of wild-type insulin. Indeed, IGF-II contains a connecting domain of 13 residues unrelated to that of proinsulin in length or sequence; the A-domain of IGF-II differs from that of proinsulin at 9 of 21 positions, and its B-domain at 18 of 30 positions. No clues are provided by comparison of the sequences of proinsulin, IGF-II or other members of the insulin-like family as guidance for the design of isoform-specific analogues.

Irrespective of theory, we have discovered that single-chain analogues of human insulin may be designed with preferential binding to IR-A with an affinity equal to or greater than that of wild-type insulin, but without enhanced binding to IGFR. Such analogues may be useful for enhancing insulin signaling through IR-A. Because signaling through IR-B is thought to mediate the hypoglycemic action of insulin, the present invention therefore allows stimulation of IR-A-dependent pathways with lower risk of adverse hypoglycemia than can be achieved by treatment with wild-type human insulin, animal insulins, and insulin analogues known in the art. In the clinical settings of Type II diabetes mellitus, the metabolic syndrome, or impaired glucose tolerance, such IR-A-dependent pathways may elicit beneficial effects on β-cell function and viability and beneficial effects on appetite control through hypothalamic circuitry and other aspects of the central nervous system. Such isoform-specific analogues may also be of value in mammalian cell culture and in experimental manipulation of wild-type and genetically modified animals.

### SUMMARY OF THE INVENTION

It is, therefore, an aspect of the present invention to provide insulin analogues that preferentially bind to IR-A relative to IR-B.

It is another aspect of the present invention to provide single-chain insulin analogues that preferentially bind to and activate IR-A relative to IR-B without enhanced binding to IGFR.

In general, the present invention provides a method of treating a mammal comprising administering a physiologically effective amount of an insulin analogue or a physiologically acceptable salt thereof where the insulin analogue displays more than twofold greater binding affinity to insulin receptor isoform A (IR-A) than insulin receptor isoform B (IR-B) and wherein the analogue has at least one third of the relative binding affinity to IR-B compared to wild type insulin from which the analogue is derived. The insulin analogue may display a binding affinity for IR-A at least fourfold, sixfold or even greater, than for IR-B.

The insulin analogue or a physiologically acceptable salt thereof may be a single-chain insulin analogue or a physiologically acceptable salt thereof, containing an insulin A-chain sequence or an analogue thereof and an insulin B-chain sequence or an analogue thereof connected by a truncated polypeptide linker compared to the linker of proinsulin. In one example, the linker may be less than 15 amino acids long. In other examples, the linker may be 4, 5, 6, 7, 8, 9, 10, 11, 12, or 13 amino acids long. In one particular example, the linker is a polypeptide having the sequence Gly-Gly-Gly-Pro-Arg-Arg (SEQ. ID. NO. 19).

In another particular example, the insulin analogue is a polypeptide having a sequence selected from the group consisting of polypeptides having the sequence of SEQ. ID. NOS. 26 and 36. In still other examples, the insulin analogue may have a sequence selected from the group consisting of polypeptides having the sequence of SEQ. ID. NO. 17, wherein Xaa₄₋₁₃ is 6 of any amino acids, with the proviso that the first two amino acids of Xaa₄₋₁₃ are not arginine. In still other examples, the insulin analogue comprises a single chain polypeptide of formula I,

B—C-A (I)

wherein B comprises a polypeptide having the sequence:

(SEQ. ID. NO. 38)
FVNQHLCGSX₂LVEALYLVCGERGFFYTX₃ X₄T

where X₂ is D or H, X₃ is P, D or K, and X₄ is K or P,

wherein C is a polypeptide consisting of the sequence GGGPRR (SEQ.ID. NO. 19), and

wherein A comprises a polypeptide having the sequence:

| | |
|---|---|
| GIVEQCCX₁SICSLYQLENYCN | (SEQ. ID. NO. 37) |

where X₁ is T or H.

In such an example, the insulin analogue may comprise a polypeptide selected from the group consisting of a polypeptide having the sequence of SEQ. ID. NO. 26 and a polypeptide having the sequence of SEQ. ID. NO. 36.

A single-chain insulin analogue of the present invention may also contain other modifications, such as substitutions of a histidine at residues A4, A8 and B1 as described more fully in co-pending International Application No. PCT/US07/00320 and U.S. application Ser. No. 12/160,187, the disclosures of which are incorporated by reference herein. In one example, the vertebrate insulin analogue is a mammalian insulin analogue, such as a human, porcine, bovine, feline, canine or equine insulin analogue.

The present invention likewise provides a pharmaceutical composition comprising such insulin analogues and which may optionally include zinc. Zinc ions may be included in such a composition at a level of a molar ratio of between 2.2 and 3.0 per hexamer of the insulin analogue. In such a formulation, the concentration of the insulin analogue would typically be between about 0.1 and about 3 mM; concentrations up to 3 mM may be used in the reservoir of an insulin pump. In another example, a pharmaceutical composition including a single-chain insulin analogue displays less than 1 percent fibrillation at 37° C. at a zinc molar ratio of less than 2, 1.5, 1 per hexamer or even in the absence of zinc other than that amount present as an impurity.

Excipients may include glycerol, glycine, other buffers and salts, and anti-microbial preservatives such as phenol and meta-cresol; the latter preservatives are known to enhance the stability of the insulin hexamer. Such a pharmaceutical composition may be used to treat a patient having diabetes mellitus or other medical condition by administering a physiologically effective amount of the composition to the patient.

The present invention also provides a nucleic acid comprising a sequence that encodes a polypeptide encoding a single-chain insulin analogue containing a sequence encoding an A chain, a B-chain and a linker between the A and B-chains containing 4-13 codons. The nucleic acid may also encode other modifications of wild-type insulin such as histidine, lysine, arginine, or other residue substitutions at residue A8 as provided in International Application No. PCT/US09/40544, the disclosure of which is incorporated by reference herein. Residues other than histidine may be substituted at position A8 or B10 to enhance stability and activity. Residues may also be substituted at positions B9, B28, and/or B29 to alter the self-association properties (and hence pharmacokinetic properties) of the analog. Residues other than tyrosine may be substituted at position A14 to adjust the isoelectric point of the analog; substitutions or additional residues may likewise be inserted within the foreshortened connecting domain to adjust the isoelectic point of the protein. The nucleic acid sequence may encode a modified A- or B-chain sequence containing an unrelated substitution or extension elsewhere in the polypeptide or modified proinsulin analogues. The nucleic acid may also be a portion of an expression vector, and that vector may be inserted into a host cell such as a prokaryotic host cell like an E. coli cell line, or a eukaryotic cell line such as Saccharomyces cerevisiae or Pischia pastoris strain or cell line.

### BRIEF DESCRIPTION OF THE SEVERAL VIEWS OF THE DRAWINGS

FIG. 1A is a schematic representation of the sequence of human proinsulin including the A- and B-chains and the connecting region shown with flanking dibasic cleavage sites (filled circles) and C-peptide (open circles). The line labeled “foreshortened connecting peptide” represents the connecting region in mini-proinsulin, which is a proinsulin analogue containing a dipeptide (Ala-Lys) linker between the A-chain and B-chain portions of insulin.

FIG. 1B is a structural model of proinsulin, consisting of an insulin-like moiety and a disordered connecting peptide (dashed line).

FIG. 2 presents results of a receptor-binding assay in which binding of the 57 mer single-chain insulin analogue (dashed line; triangles) was evaluated relative to native human insulin (solid line; squares). This assay measures the displacement of receptor-bound ¹²⁵I-labeled insulin by either unlabeled analogue or cold insulin. (A, top panel) Binding of insulin or insulin analogue to IR-A. (B, middle panel) Binding of insulin or insulin analogue to IR-B. (C, bottom panel) Binding of insulin or insulin analogue to IGFR.

FIG. 3A is a graph of the results of a receptor binding assay in which binding of human insulin and human insulin analogues to human insulin receptor isoform A (HIRA) were evaluated. The displacement of receptor-bound ¹²⁵I-labeled insulin by either unlabeled analogue or insulin (B/Bo) is provided across a range of unlabeled analog/insulin concentrations.

FIG. 3B is a graph of the results of a receptor binding assay in which binding of human insulin and human insulin analogues to human insulin receptor isoform B (HIRB) were evaluated. The displacement of receptor-bound ¹²⁵I-labeled insulin by either unlabeled analogue or insulin (B/Bo) is provided across a range of unlabeled analog/insulin concentrations.

FIG. 3C is a graph of the results of a receptor binding assay in which binding of human insulin and human insulin analogues to Insulin-like Growth Factor Receptor (IGFR) were evaluated. The displacement of receptor-bound ¹²⁵I-labeled insulin by either unlabeled analogue or insulin (B/Bo) is provided across a range of unlabeled analog/insulin concentrations.

FIG. 4 is a graph of the results of a receptor binding assay comparing the IGFR binding affinity of a single chain insulin (SCI) that is wild type at position B10 (SEQ. ID. NO. 26), with Insulin-like Growth Factor 1 (IGF-1), wild type human insulin and the insulin analogues sold under the trademarks Humalog® and Lantus®.

FIG. 5 is a graph showing blood sugar measurements of diabetic Lewis rats over time following injection of human insulin (SEQ. ID. NOS. 2 and 3), SCI (His^{A8}, Asp^{B10}, Asp^{B28}, and Pro^{B29}) (SEQ. ID. NO. 36), or a double stranded analog of the SCI (having the His^{A8}, Asp^{B10}, Asp^{B28}, and Pro^{B29} substitutions) (SEQ. ID. NOS. 34 and 35).

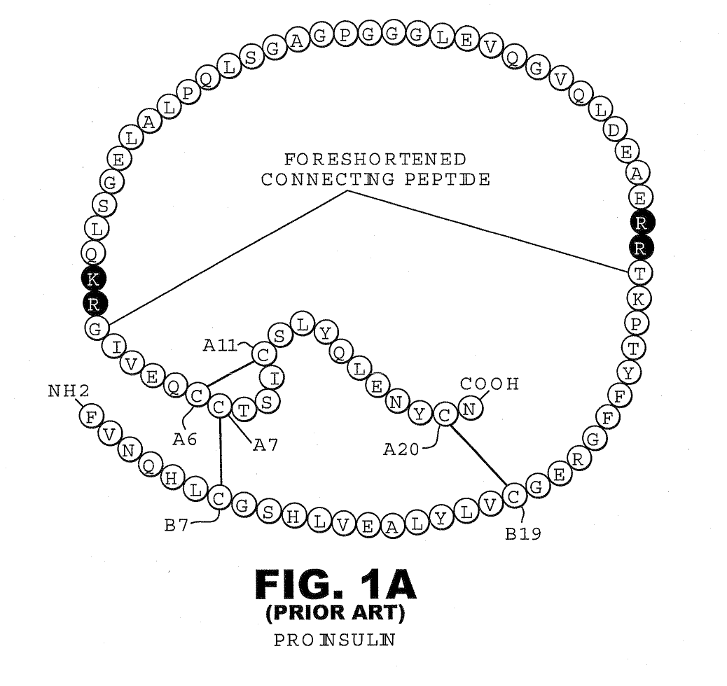

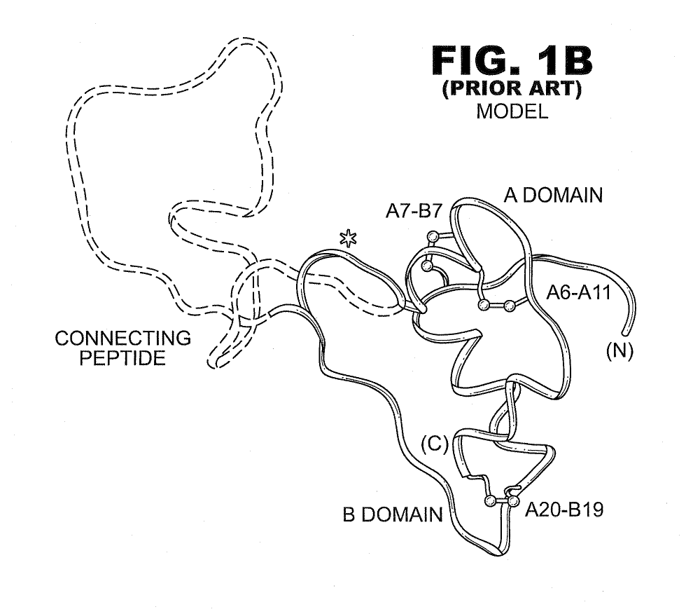

### DETAILED DESCRIPTION OF THE INVENTION

The present invention is directed toward recombinant single-chain insulin analogues that provide isoform-specific binding of the analogue to the A-isoform of the insulin receptor (IR-A) with binding to the B-isoform (IR-B) reduced by at least sixfold. To that end, the present invention provides insulin analogues that contain a variant insulin A-chain polypeptide and a variant insulin B-chain polypeptide connected by a truncated linker polypeptide. In one example, the linker polypeptide may be less than 15 amino acids long. In other examples, the linker polypeptide may be 4, 5, 6, 7, 8, 9, 10, 11, 12, or 13 amino acids long.

The single-chain insulin analogue of the present invention may also contain other modifications. As used in this specification and the claims, various substitution analogues of insulin may be noted by the convention that indicates the amino acid being substituted, followed by the position of the amino acid, optionally in superscript. The position of the amino acid in question includes the A or B-chain of insulin where the substitution is located. For example, the single-chain insulin analogue of the present invention may also contain a substitution of aspartic acid (Asp or D) or lysine (Lys or K) for proline (Pro or P) at amino acid 28 of the B-chain (B28), or a substitution of Pro for Lys at amino acid 29 of the B-chain (B29) or a combination thereof. These substitutions may also be denoted as Asp^{B28}, Lys^{B28}, and Pro^{B29}, respectively. Unless noted otherwise or wherever obvious from the context, the amino acids noted herein should be considered to be L-amino acids.

Another aspect of this invention is avoidance of significantly increased cross-binding to the IGF Type I receptor. To that end, it may be advantageous to utilize a linker that does not contain the sequence Arg-Arg-Xaa or a tyrosine with tandem arginines as present in the Insulin-like Growth Factor I (IGF-1) C-domain because these sequences have been identified as being important for binding of IGF-1 to IGFR.

The Asp^{B28} substitution is present in the insulin analogue known as Aspart insulin and sold as Novalog® whereas the Lys^{B28} and Pro^{B29} substitutions are present in the insulin analogue known as Lispro insulin and sold under the name Humalog®. These analogues are described in U.S. Pat. Nos. 5,149,777 and 5,474,978, the disclosures of which are hereby incorporated by reference herein. Both of these analogues are known as fast-acting insulins. Neither of these analogues exhibits isoform-specific receptor binding.

It is further envisioned that the single-chain insulin analogues of the present invention may also utilize any of a number of changes present in existing insulin analogues, modified insulins, or within various pharmaceutical formulations, such as regular insulin, NPH insulin, lente insulin or ultralente insulin, in addition to human insulin. The single-chain insulin analogues of the present invention may also contain substitutions present in analogues of human insulin that, while not clinically used, are still useful experimentally, such as DKP-insulin, which contains the substitutions Asp^{B10}, Lys^{B28} and Pro^{B29} or the Asp^{B9} substitution. The present invention is not, however, limited to human insulin and its analogues. It is also envisioned that these substitutions may also be made in animal insulins such as porcine, bovine, equine, and canine insulins, by way of non-limiting examples. Furthermore, in view of the similarity between human and animal insulins, and use in the past of animal insulins in human diabetic patients, it is also envisioned that other minor modifications in the sequence of insulin may be introduced, especially those substitutions considered “conservative” substitutions. For example, additional substitutions of amino acids may be made within groups of amino acids with similar side chains, without departing from the present invention. These include the neutral hydrophobic amino acids: Alanine (Ala or A), Valine (Val or V), Leucine (Leu or L), Isoleucine (Ile or I), Proline (Pro or P), Tryptophan (Trp or W), Phenylalanine (Phe or F) and Methionine (Met or M). Likewise, the neutral polar amino acids may be substituted for each other within their group of Glycine (Gly or G), Serine (Ser or S), Threonine (Thr or T), Tyrosine (Tyr or Y), Cysteine (Cys or C), Glutamine (Glu or Q), and Asparagine (Asn or N). Basic amino acids are considered to include Lysine (Lys or K), Arginine (Arg or R) and Histidine (His or H). Acidic amino acids are Aspartic acid (Asp or D) and Glutamic acid (Glu or E). In one example, the insulin analogue of the present invention contains three or fewer conservative substitutions other than the modified linker of the present invention.

The amino acid sequence of human proinsulin is provided, for comparative purposes, as SEQ. ID. NO. 1. The amino-acid sequence of the A-chain of human insulin is provided as SEQ. ID. NO. 2. The amino acid sequence of the B-chain of human insulin is provided, for comparative purposes, as SEQ. ID. NO. 3.

SEQ. ID. NO. 1
(proinsulin)
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Arg-Arg-Glu-Ala-Glu-Asp-
Leu-Gln-Val-Gly-Gln-Val-Glu-Leu-Gly-Gly-Gly-Pro-
Gly-Ala-Gly-Ser-Leu-Gln-Pro-Leu-Ala-Leu-Glu-Gly-
Ser-Leu-Gln-Lys-Arg-Gly-Ile-Val-Glu-Gln-Cys-Cys-
Thr-Ser-Ile-Cys-Ser-Leu-Tyr-Gln-Leu-Glu-Asn-Tyr-
Cys-Asn
SEQ. ID. NO. 2
(A chain)
Gly-Ile-Val-Glu-Gln-Cys-Cys-Thr-Ser-Ile-Cys-Ser-
Leu-Tyr-Gln-Leu-Glu-Asn-Tyr-Cys-Asn
SEQ. ID. NO. 3
(B-chain)
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr

The amino-acid sequence of a single-chain human insulin of the present invention is provided as SEQ. ID. NO. 4, where Xaa represents any amino acid.

SEQ. ID. NO. 4
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Xaa₄₋₁₃-Gly-Ile-Val-Glu-
Gln-Cys-Cys-Thr-Ser-Ile-Cys-Ser-Leu-Tyr-Gln-Leu-
Glu-Asn-Tyr-Cys-Asn

In various examples, the linker represented by Xaa may be 4, 5, 6, 7, 8, 9, 10, 11, 12, or 13 amino acids in length. In one example, the linker comprises the naturally occurring amino acids that immediately flank the A and B-chains. SEQ. ID. NOS. 5-14 provide sequences where the linker comprises amino acids in their naturally occurring locations in proinsulin. Stated another way, the natural linker of proinsulin is truncated in varying amounts, leaving amino acids naturally found immediately adjacent to the A- and B-chains in proinsulin. In SEQ. ID. NO. 5, the Arg residues immediately flanking the A- and B-chains are present. In SEQ. ID. NO. 6, the two Arg residues normally found adjacent the B-chain and the Arg and Lys residues normally found adjacent the A chain are present. In SEQ. ID. NOS. 7 and 8, the Arg-Arg-Glu sequence normally found adjacent the B-chain and the Gln-Lys-Arg sequence normally found adjacent the A chain are present. In SEQ. ID. NO. 7 an additional 1-4 amino acids may optionally be present.

SEQ. ID. NO. 5
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Arg-Xaa₂₋₈-Arg-Gly-Ile-
Val-Glu-Gln-Cys-Cys-Thr-Ser-Ile-Cys-Ser-Leu-Tyr-
Gln-Leu-Glu-Asn-Tyr-Cys-Asn
SEQ. ID. NO. 6
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Arg-Arg-Xaa₀₋₆-Lys-Arg-
Gly-Ile-Val-Glu-Gln-Cys-Cys-Thr-Ser-Ile-Cys-Ser-
Leu-Tyr-Gln-Leu-Glu-Asn-Tyr-Cys-Asn
SEQ. ID. NO. 7
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Arg-Arg-Glu-Xaa₀₋₄-Gln-
Lys-Arg-Gly-Ile-Val-Glu-Gln-Cys-Cys-Thr-Ser-Ile-
Cys-Ser-Leu-Tyr-Gln-Leu-Glu-Asn-Tyr-Cys-Asn
SEQ. ID. NO. 8
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Arg-Arg-Glu-Gln-Lys-Arg-
Gly-Ile-Val-Glu-Gln-Cys-Cys-Thr-Ser-Ile-Cys-Ser-
Leu-Tyr-Gln-Leu-Glu-Asn-Tyr-Cys-Asn

SEQ. ID. NOS. 9-14 provide linkers of varying lengths, consisting of various sequences found naturally in the sequence of proinsulin.

Other truncated linkers, with sequences not found naturally in insulin, may also be utilized. For example, SEQ. ID. NO. 19 provides a linker having the sequence Gly-Gly-Gly-Pro-Arg-Arg, SEQ. ID. NO. 20 provides a linker having the sequence Gly-Gly-Pro-Arg-Arg, SEQ. ID. NO. 21 provides a linker having the sequence Gly-Ser-Glu-Gln-Arg-Arg, SEQ. ID. NO. 22 provides a linker having the sequence Arg-Arg-Glu-Gln-Lys-Arg, SEQ. ID. NO. 23 provides a linker having the sequence Arg-Arg-Glu-Ala-Leu-Gln-Lys-Arg, SEQ. ID. NO. 24 provides a linker having the sequence Gly-Ala-Gly-Pro-Arg-Arg, and SEQ. ID. NO. 25 provides a linker having the sequence Gly-Pro-Arg-Arg. It is envisioned that any of these truncated linkers may be used in a single-chain insulin analogue of the present invention, either alone or in combination with other substitutions or other changes in the insulin polypeptide sequence as noted herein.

Various substitutions, including substitutions of prior known insulin analogues, may also be present in the single-chain insulin analogue of the present invention. For example, an amino-acid sequence of a single-chain insulin analogue also carrying substitutions corresponding to the Lys^{B28} Pro^{B29} substitutions of lispro insulin is provided as SEQ. ID. NO. 15. Likewise, an amino acid sequence of a single-chain insulin analogue also carrying substitutions corresponding to the Asp^{B28} substitution of aspart insulin is provided as SEQ. ID. NO. 16. Additionally, exemplary amino acid sequences of single-chain insulin analogues also carrying substitutions corresponding to the Asp^{B10} substitution are provided as SEQ. ID. NOS. 17 and 18.

SEQ. ID. NO. 15
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Lys-Pro-Thr-Xaa₄₋₁₀-Gly-Ile-Val-Glu-
Gln-Cys-Cys-Thr-Ser-Ile-Cys-Ser-Leu-Tyr-Gln-Leu-
Glu-Asn-Tyr-Cys-Asn
SEQ. ID. NO. 16
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Asp-Thr-Xaa₄₋₁₀-Gly-Ile-Val-Glu-
Gln-Cys-Cys-Thr-Ser-Ile-Cys-Ser-Leu-Tyr-Gln-Leu-
Glu-Asn-Tyr-Cys-Asn
SEQ. ID. NO. 17
Phe-Val-Asn-Gln-His-Leu-Cys-Gly-Ser-Asp-Leu-Val-
Glu-Ala-Leu-Tyr-Leu-Val-Cys-Gly-Glu-Arg-Gly-Phe-
Phe-Tyr-Thr-Pro-Lys-Thr-Xaa₄₋₁₀-Gly-Ile-Val-Glu-
Gln-Cys-Cys-Thr-Ser-Ile-Cys-Ser-Leu-Tyr-Gln-Leu-
Glu-Asn-Tyr-Cys-Asn
SEQ. ID. NO. 18
Phe-Val-Asn-Gln-His-Leu-Cys-Gly-Ser-Asp-Leu-Val-
Glu-Ala-Leu-Tyr-Leu-Val-Cys-Gly-Glu-Arg-Gly-Phe-
Phe-Tyr-Thr-Pro-Lys-Thr-Arg-Arg-Glu-Xaa₀₋₄-Gln-
Lys-Arg-Gly-Ile-Val-Glu-Gln-Cys-Cys-Thr-Ser-Ile-
Cys-Ser-Leu-Tyr-Gln-Leu-Glu-Asn-Tyr-Cys-Asn

The activities of insulin or insulin analogues may be determined by receptor binding assays as described in more detail herein below. Relative activity may be defined by comparison of the dissociation constants (K_{eq}) governing the hormone-receptor binding reaction. Relative activity may also be estimated by comparison of ED₅₀ values, the concentration of unlabelled insulin or insulin analogue required to displace 50 percent of specifically bound labeled human insulin, such as a radioactively-labeled human insulin (such as ¹²⁵I-labeled insulin) or a radioactively-labeled high-affinity insulin analog. Alternatively, activity may be expressed simply as a percentage of the activity of normal insulin. Affinity for the insulin-like growth factor receptor (IGFR) may also be determined in the same way with displacement of a radioactively labeled IGF-1 (such as ¹²⁵I-labeled IGF-1) from IGFR being measured. In particular, it is desirable for an isoform-selective single-chain insulin analogue to have an activity that is equal to or greater than 100 percent of insulin for one isoform of the insulin receptor, such as 110, 120, 130, 140, 150, or 200 percent of normal insulin or more, while having an affinity for the other isoform of the insulin receptor that is reduced by at least sixfold relative to the targeted isoform. It is also desirable that cross-binding of the single-chain insulin analogue to the IGFR is less than or equal to 100 percent of normal insulin, such as 90, 80, 70, 60 or 50 percent of normal insulin or less. It is desirable to determine insulin activity in vitro as described herein, rather than in vivo. It has been noted that in vivo, clearance of insulin from the bloodstream is dependent on receptor binding. In this way, insulin analogues may exhibit high activity over several hours, even approaching approximately 100 percent activity in vivo, even though they are less active at the cellular level, due to slower clearance from the bloodstream. However, an insulin analogue can still be useful in the treatment of diabetes even if the in vitro receptor-binding activity is as low as 20% due to this slower clearance and the feasibility of administration of higher doses.

A single-chain analogue of insulin was made by total chemical synthesis using thiol-ester-mediated native fragment ligation of three polypeptide segments. The segments comprised residues 1-18 (segment I), 19-42 (segment II), and 43-57 (segment III). Each segment was synthesized by the solid-phase method. Segments I and segment II were prepared by N-α-tert-butyloxycarbonyl (Boc)-chemistry on OCH₂-Pam resin(Applied Biosystems); segment III was prepared by N-α-(9-fluoronylmethoxycarbonyl (Fmoc)-chemistry on Polyethylene Glycol-Polystyrene (PEG-PS) resin with standard side-chain protecting groups. Segment I was synthesized as a thioester (beta-mercaptoleucine, βMp-Leu). The synthesis was started from Boc-Leu-OCH₂-Pam resin, and the peptide chain was extended stepwise to the N-terminal residue. Segment II was also synthesized as a thioester with peptide, Arg-Arg-Gly, attached at the C-terminal of βMp-residue to enhance solubility of the segment. The N-terminal amino acid, Cysteine, of segment II was protected as thiazolidine (Thz) and converted to Cysteine by MeONH₂.HCl after the ligation. Following native ligation, the full-length polypeptide chain was allowed to fold in a mixture of 100 mM reduced glutathione (GSH) and 10 mM oxidized glutathione (GSSG) at pH 8.6 and subjected to HPLC purification using C4 column (1.0×25 cm) at the gradient elution from 15% to 35% (A/B) over 40 min at the flow rate of 4 ml/min. The pure fractions corresponding to SCI (1) were pooled and freeze-dried. The predicted molecular mass was verified by mass spectrometry.

A single-chain insulin analogue having the polypeptide sequence of SEQ. ID. NO. 26 was prepared.

SEQ. ID. NO. 26
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Asp-Pro-Thr-Gly-Gly-Gly-Pro-Arg-Arg-
Gly-Ile-Val-Glu-Gln-Cys-Cys-His-Ser-Ile-Cys-Ser-
Leu-Tyr-Gln-Leu-Glu-Asn-Tyr-Cys-Asn

This 57-mer single-chain analogue was synthesized and tested for activity. This analogue contains a modified A-chain sequence (containing the substitution His^{A8}) and a modified B-chain sequence (containing the substitutions Asp^{B28} and Pro^{B29}) with 6-residue linker of sequence GGGPRR. For comparative purposes, a 58-mer single-chain insulin analogue was likewise prepared containing the sequence previously described by Lee and colleagues (Nature, Vol. 408, pp 483-488, 2000). The latter analogue contains wild-type A-chain and B-chain sequences with 7-residue linker of sequence GGGPGKR (SEQ. ID. NO. 33, “Prior SCI”). It should be noted, however, that the results described in the article describing this analogue have recently been withdrawn by at least some of the authors of the original article (Nature, Vol. 458, p. 660, 2009), casting doubt on the validity of the results as presented in the original Nature article. Nevertheless, a comparison between a single chain insulin according to the present invention and the prior single chain insulin is presented herein.

Synthetic genes were synthesized to direct the expression of the same polypeptide in yeast Piscia pastoris and other microorganisms. The sequence of the DNA is either of the following:

(a) with Human Codon preferences
(SEQ. ID. NO. 28)
TTC/GTC/AAC/CAG/CAC/CTC/TGC/GGC/AGC/CAC/CTC/GTC/
GAA/GCA/CTC/TAC/CTC/GTC/TGC/GGA/GAA/CGA/GGA/TTC/
TTC/TAC/ACA/GAC/CCA/ACA/GGA/GGA/GGA/CCA/CGA/CGA/
GGA/ATA/GTA/GAA/CAA/TGC/TGC/CAC/AGC/ATA/TGT/AGC/
CTC/TAC/CAA/CTA/GAA/AAC/TAC/TGC/AAC
(b) with Pichia Codon Preferences
(SEQ. ID. NO. 29)
TTT/GTT/AAC/CAA/CAT/TTG/TGT/GGT/TCT/GAT/TTG/GTT/
GAA/GCT/TTG/TAC/TTG/GTT/TGT/GGT/GAA/AGA/GGT/TTT/
TTT/TAC/ACT/GAT/CCA/ACT/GGT/GGT/GGT/CCA/AGA/AGA/
GGT/ATT/GTT/GAA/CAA/TGT/TGT/CAT/TCT/ATT/TGT/TCT/
TTG/TAC/CAA/TTG/GAA/AAC/TAC/TGT/AAC

Other variants of these sequences, encoding the same polypeptide sequence, are possible given the synonyms in the genetic code. Additional synthetic genes were prepared to direct the synthesis of analogues of this polypeptide containing variant amino-acid substitutions at positions A4, A8, B28 and B29; in addition, successive changes in length of the linker peptide were encoded within the variant DNA sequence.

Receptor-Binding Assays. Relative activity is defined as the ratio of dissociation constants between the analogue and wild-type human insulin as determined by competitive binding assays using ¹²⁵I-human insulin as a tracer. This assay employs the purified epitope-tagged receptor (IR-A, IR-B, or IGFR) using a microtiter-plate antibody-capture assay as known in the art. The epitope tag consists of three tandem repeats of the FLAG epitope. Microtiter strip plates (Nunc Maxisorb) were incubated overnight at 4° C. with anti-FLAG IgG (100 μl/well of 40 mg/ml in phosphate-buffered saline). Binding data were analyzed by a single-site heterologous competition binding model. A corresponding microtiter plate antibody assay using the epitope-tagged IGF Type I receptor was employed to assess cross-binding of analogues to this homologous receptor. In all assays the percentage of tracer bound in the absence of competing ligand was less than 15% to avoid ligand-depletion artifacts.

Relative affinities for IR-A and IR-B are provided in Table 1; values are normalized to 100%, defined by the binding affinity of wild-type human insulin for IR-A. The affinity of human insulin is 0.04 nM under assay conditions. Corresponding affinities for IGFR are given in column 4; the affinity of human insulin for IGFR is 9.7 nM under assay conditions.

TABLE I

| | RELATIVE AFFINITY | | |
|---|---|---|---|
| | INSULIN | | |
| | RECEPTOR | | IGF-I |
| LIGAND | Isoform A | Isoform B | RECEPTOR |
| Insulin (SEQ. ID. NOS. 2 and 3) | 100 | 72 | 0.3 |
| Proinsulin (SEQ. ID. NO. 1) | 4 | 1 | ND |
| IGF-I | 2 | 0.7 | 1700 |
| IGF-II | 15 | 4 | 140 |
| Humalog ® | 109 | 67 | 0.3 |
| Novalog ® | 147 | 85 | 0.6 |
| Trp^{A13}-KP insulin (SEQ. ID. NOS. | 85 | 49 | ND |
| 3 and 31) | | | |
| Trp^{A14}-insulin (SEQ. ID. NOS. 3 | 170 | 60 | ND |
| and 32) | | | |
| Prior-SCI (SEQ. ID. NO. 33) | 5 | 3 | 0.1 |
| [His^{A8,} Asp^{B28}, Pro^{B29}]-SCI (SEQ. | 200 | 26 | 0.1 |
| ID. NO. 26) | | | |

As expected, wild-type insulin exhibits a small preference for IR-A relative to IR-B (row 1 in Table I). A similarly small preference for IR-A is observed in studies of Humalog® and Novalog® (rows 5 and 6). Substitutions in the middle of the A-chain (replacement of Leu^{A13} or Tyr^{A14} by Trp; rows 7 and 8, respectively) likewise confer less than twofold selectivity for IR-A. Although the single-chain ligands proinsulin, IGF-1, and IGF-II each bind poorly to either isoform of the insulin receptor, these ligands exhibit greater than twofold preference for IR-A (rows 2-4 in Table I).

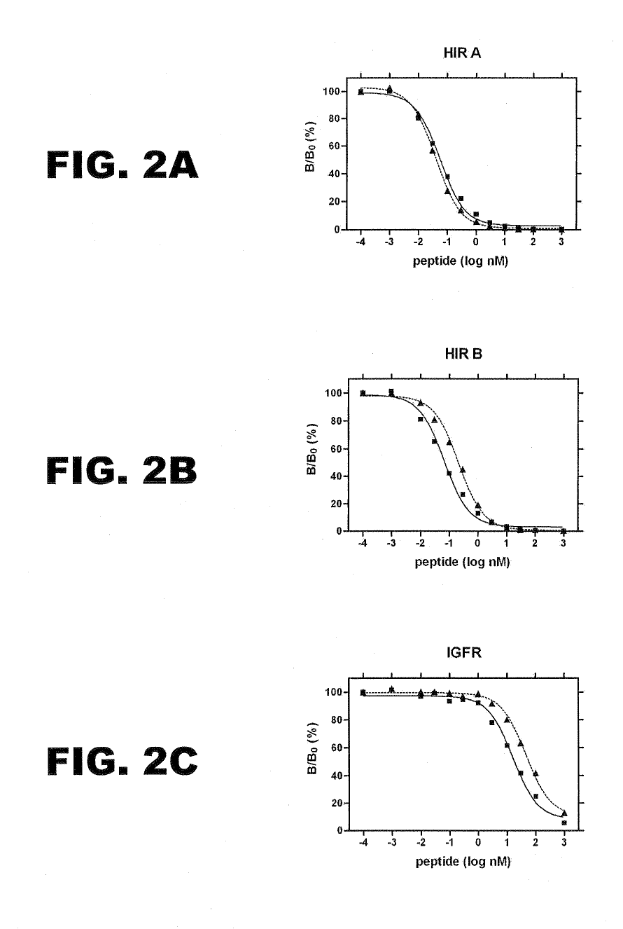

The IR-A receptor-binding activity of the 57 mer single-chain insulin analogue (SEQ. ID. NO. 26) relative to human insulin is 200%, as shown in Table I (bottom row); its affinity for IR-B is less than 30%, and its affinity for IGFR is threefold lower than that of human insulin. These binding properties are illustrated in FIG. 2 by a set of receptor-binding assays in which binding of the 57 mer single-chain insulin analogue (dashed line; triangles) was evaluated relative to native human insulin (solid line; squares): (A) binding to IR-A, (B) binding to IR-B, and (C) binding to IGFR. These assays measure the displacement of receptor-bound ¹²⁵I-labeled insulin by either unlabeled analogue or insulin (B/Bₒ) across a range of unlabeled analog/insulin concentrations.

Control studies of a single-chain insulin known in the art (Prior-SCI; second row from bottom in Table I) demonstrates that it binds with low affinity to either isoform of the insulin receptor and without significant in change in isoform selectivity relative to human insulin.

The in vivo potency of the 57 mer SCI containing His^{A8}, Asp^{B28}, and Pro^{B29} substitutions (SEQ. ID. NO. 26) in diabetic rats was evaluated relative to wild-type human insulin (SEQ. ID. NOS. 2 and 3). To this end, male Lewis rats (˜250 g body weight) were rendered diabetic with streptozotocin. Human insulin and the SCI were purified by HPLC, dried to powder, and dissolved in insulin diluent (Eli Lilly Corp). Rats were injected subcutaneously at time=0 with a range of insulin doses from 0-1.5 U/kg body weight (typically to 0-30 micrograms of protein per rat) in 100 μl of diluent; corresponding aliquots of SCI were prepared based on moles of protein. Blood was obtained from clipped tip of the tail at time 0 and every 10 minutes up to 90 min. Blood glucose was measured using a Hypoguard Advance Micro-Draw meter. At submaximal concentrations of insulin, three-fold higher molar concentrations of the SCI were required to achieve the same rate and extent of blood glucose lowering as wild-type insulin. The higher dose of SCI needed on a molar basis is in accord with its ca. threefold lower binding affinity for the B isoform of the insulin receptor, as it is the B isoform that is thought to mediate hormone-dependent glucose uptake into target tissues. For wild type human insulin, the mean change in blood glucose (6 rats) was approximately −115.6 mg/dL per hour following a dose of 0.5 U/kg (a submaximal dose). For the SCI at the same dose in moles of protein, the mean change in blood glucose was −31.4 mg/dL per hour, almost fourfold lower. When the amount of SCI injected was increased to the weight equivalent of 1.5 U/kg, a mean drop in blood glucose of −98.7 mg/dL per hour was observed. This indicates that the full potency of the analogue for blood glucose control can be achieved by increasing the molar amount injected. At such a dose, a patient can control his or her blood glucose but obtain increased activation of the A-isoform signaling pathway. Such differentiated signaling may selectively affect the beta cells and/or the brain.

The isoform-selective activity of SCI was evaluated in relation to wild-type insulin using IGFR^{-/-} murine fibroblasts stably transfected to express either insulin receptor isoform A or insulin receptor isoform B. These cell lines exhibit negligible background expression of the murine insulin receptor but contain insulin receptor substrate 1 (IRS-1). Cells were grown to ˜80% confluency, serum-starved overnight, and treated with 10 nM wild-type human insulin (Sigma) or SCI for 5 minutes. Following immunoprecipitation of the insulin receptor, ligand-dependent autophosphorylation of the receptor was probed by Western blot using an anti-phosphotyrosine antiserum (PY20). Blots were stripped and reprobed with the anti-receptor antibody to enable correction for extent of isoform-specific receptor expression. SCI activated receptor isoform A at least as efficiently as wild-type insulin. By striking contrast, SCI-dependent autophosphorylation of receptor isoform B was 47±11 percent less efficient than was insulin-dependent authophosphylation of receptor isoform B. These data show that the isoform-specific receptor-binding properties of SCI in vitro correspond to isoform-specific receptor activation in a cellular context. Analogous Western blots to probe for extent of ligand-dependent phosphorylation of IRS-1 similarly demonstrate proportionate isoform-specific signaling by SCI.

The receptor binding activity of another analogue according to the present invention was also compared to the analogue of SEQ. ID. NO. 33 (“Prior SCI”). Single chain insulin analogues (SCI) of the invention containing His^{A8}, Asp^{B28}, and Pro^{B29} substitutions with (SEQ. ID. NO. 36) or without (SEQ. ID. NO. 26) an Asp^{B10} substitution were compared. In Table II, the binding affinities for wild type human insulin (HI) and several insulin analogues for the A isoform specific human insulin receptor (HIRA), the β isoform specific human insulin receptor (HIRB), and Insulin-like Growth Factor receptor (IGFR) are provided. The Prior SCI had greatly reduced affinity for insulin receptors compared to human insulin. The insulin analogue indicated as “A8-His, B-10 Asp, B 28-Asp, B 29-Pro ins” has the sequences of SEQ. ID. NOS. 34 and 35.

The affinities of the insulin analogues to HIRA, HIRB and IGFR are provided as dissociation constants (Kd) and as an absolute number relative to unmodified human insulin. The prior SCI had affinities for HIRA and HIRB of 5 percent and 4 percent of human insulin respectively. Affinity of the prior SCI for IGFR relative to human insulin was greater, but was still only 13 percent of human insulin. The SCI containing the substitution Asp^{B10} (SEQ. ID. NO. 36) has an affinity for the A isoform insulin receptor approximately 7 fold greater than that of human insulin and an affinity for the β isoform insulin receptor of about half that of human insulin. At the same time, the affinity of this SCI for IFGR is approximately the same as that of human insulin. By way of contrast, the SCI not containing the Asp^{B10} substitution (SEQ. ID. NO. 26) had a reduced affinity for IFGR (0.35 relative to human insulin) but also had lower affinities for HIRA and HIRB compared to the SCI containing the Asp^{B10} substitution (2.0 and 0.36, respectively). The corresponding two chain analogue, that is, the two chain analogue containing the substitutions Asp^{B10}, His^{A8}, Asp^{B28} and Pro^{B29} (SEQ. ID. NOS. 34 and 35), had an increased affinity for IFGR (3.54) over that of human insulin as well as increased affinities for HIRA and HIRB (4.25 and 4.7, respectively). The present invention therefore, provides an insulin analogue containing an Asp^{B10} substitution that maintains at least half of the affinity of human insulin for HIRB and has greater affinity for HIRA than human insulin while maintaining the affinity for IFGR at approximately the same level as unmodified human insulin.

TABLE II

| | RECEPTOR | | | | | |
|---|---|---|---|---|---|---|
| | HIRA | | HIRB | | IGFR | |
| | | Relative | | Relative | | Relative |
| LIGAND | Kd (nM) | Affinity | Kd (nM) | Affinity | Kd (nM) | Affinity |
| Human Insulin (wt) | 0.034 ± 0.002 | 1 | 0.047 ± 0.003 | 1 | 9.57 ± 0.31 | 1 |
| His^{A8}, Asp^{B10}, | 0.008 ± 0.001 | 4.25 | 0.010 ± 0.001 | 4.7 | 2.7 ± 0.003 | 3.54 |
| Asp^{B28}, Pro^{B29} | | | | | | |
| insulin | | | | | | |
| His^{A8}, Asp^{B28}, | 0.017 ± 0.001 | 2.0 | 0.130 ± 0.001 | 0.36 | 27.63 ± 1.18 | 0.35 |
| Pro^{B29} SCI | | | | | | |
| His^{A8}, Asp^{B10}, | 0.005 ± 0.0003 | 6.8 | 0.093 ± 0.003 | 0.5 | 9.89 ± 0.035 | 0.97 |
| Asp^{B28}, Pro^{B29} | | | | | | |
| SCI | | | | | | |
| Prior SCI | 0.66 ± 0.08 | 0.05 | 1.28 ± 0.15 | 0.04 | 77.4 ± 15.5 | 0.13 |

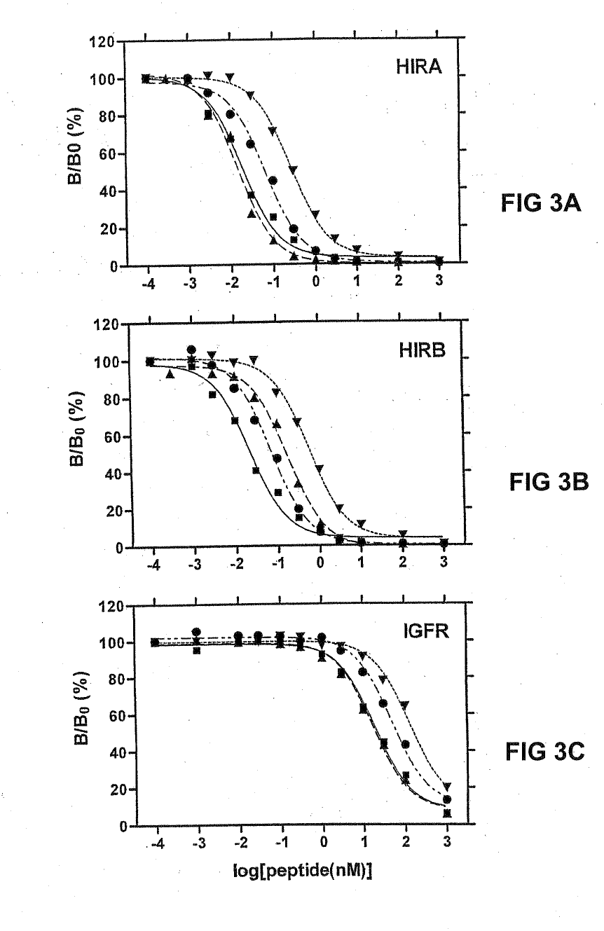

This is confirmed by the results of the receptor-binding assays shown in FIGS. 3A-3C. The insulin and insulin analogue data are represented as follows: unmodified human insulin (▪), single chain insulin (SCI) analogue containing His^{A8}, Asp^{B10}, Asp^{B28}, Pro^{B29} substitutions (▴), SCI analogue containing His^{A8}, Asp^{B28}, Pro^{B29} substitutions (), Prior SCI (▾). In FIG. 3A, the receptor-binding assay utilized HIRA. In FIG. 3B, the receptor binding assay utilized HIRB and in FIG. 3C the receptor-binding assay utilized tested. These assays measure the displacement of receptor-bound ¹²⁵I-labeled insulin by either unlabeled analogue or insulin (B/Bo) across a range of unlabeled analog/insulin concentrations.

Table III provides the binding affinities for Insulin-like Growth Factor 1 (IGF-1), wild type human insulin (HI), a single chain insulin (SCI) having the amino acid sequence of SEQ. ID. NO. 26 (His^{A8}, Asp^{B28}, Pro^{B29}) and insulin analogues Humalog® (Lys^{B28}, Pro^{B29}) and Lantus (having the addition of two arginine residues attached to the carboxy-terminal end of the B-chain). The affinities of these ligands to IGFR are provided as dissociation constants (Kd) and as an absolute number relative to IGF-1. While the SCI of the present invention shows an affinity for IGFR that is less than that of wild type insulin, the analogues Humalog® and Lantus® have affinities approximately 2-3 times that of unmodified human insulin.

TABLE III

IGFR

| LIGAND | Kd (nM) | Relative Affinity |
|---|---|---|
| IGF-I | 0.047 ± 0.006 | 1 |
| HI | 9.57 ± 0.31 | 0.005 |
| Humalog ® | 5.18 ± 0.18 | 0.009 |
| His^{A8}, Asp^{B28}, Pro^{B29} SCI | 27.63 ± 1.18 | 0.002 |
| Lantus ® | 3.14 ± 0.44 | 0.015 |

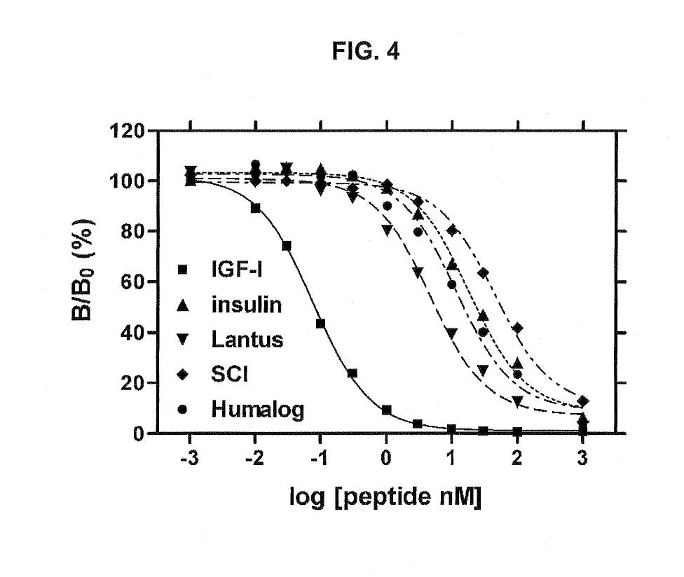

This is also reflected in FIG. 4, which is a graph showing the displacement of receptor-bound ¹²⁵I-labeled IGF-1 by unlabeled ligand (B/Bo) across a range of unlabeled peptide concentrations.

While not wishing to be bound by theory, the Applicant believes that the reduced binding activity of the prior SCI is due to an altered isoelectric point caused by the presence of lysine and arginine in the linker without an offsetting substitution in the A- or B-chain to retain. The single chain insulin analog of SEQ. ID. NO. 36, however, has a similar isoelectric point to that of human insulin, as the positive charges provided by the residues introduced in the linker offset at least some of the altered charges introduced by the Asp^{B10}, Asp^{B28} and Pro^{B29} substitutions. Additional or alternate substitutions in the A- or B-chains may also be utilized to affect the isoelectric point of a resulting insulin analog. For example, histidine may be maintained at B 10 to maintain zinc binding and insulin hexamer formation.

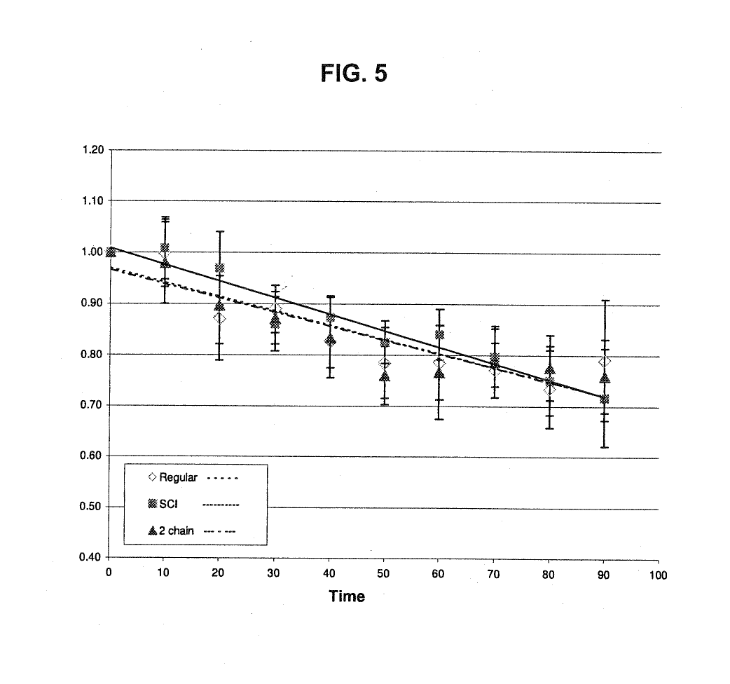

The in vivo potency of the 57 mer SCI containing His^{A8}, Asp^{B10}, Asp^{B28}, and Pro^{B29} substitutions (SEQ. ID. NO. 36) in diabetic rats is equivalent to wild-type human insulin. Male Lewis rats (˜250 g body weight) were rendered diabetic with streptozotocin. Human insulin and insulin analogs (SCI (SEQ. ID. NO. 36) and a two-chain analogue of the SCI lacking the 6-residue linker (SEQ. ID. NOS. 34 and 35)) were purified by HPLC, dried to powder, and dissolved in insulin diluent (Eli Lilly Corp). Rats were injected subcutaneously at time=0 with 1.5 U/kg body weight in 100 μl of diluent. Blood was obtained from clipped tip of the tail at time 0 and every 10 minutes up to 90 min. Blood glucose was measured using a Hypoguard Advance Micro-Draw meter. Blood glucose concentrations were observed to decrease at rates of 64.2±16.9, 62.0±16.3, and 53.2±11.7 mg/dL per h for human insulin, SCI, and the two-chain control analog, respectively. These values are indistinguishable within variation (FIG. 5). In FIG. 5, the relative blood glucose level over time is shown for human insulin (o), SCI (His^{A8}, Asp^{B10}, Asp^{B28}, and Pro^{B29}) (▪), two-chain analogue (His^{A8}, Asp^{B10}, Asp^{B28}, and Pro^{B29}) (▴). In full dose-response curves, SCI (His^{A8}, Asp^{B10}, Asp^{B28}, and Pro^{B29}) is likewise indistinguishable in its hypoglycemic action from wild-type human insulin.

Use of Asp^{B10} has previously been avoided in insulin analog formulations in clinical use due to its effect on cross-binding to the IGFR and associated mitogenicity. Testing of Asp^{B10}-insulin in Sprague-Dawley rats led to an increased incidence of mammary tumors. IGF-1 contains a negative charge at the homologous position (Glu9); it is believed that mimicry of this charge by Asp^{B10} significantly enhances the binding of Asp^{B10}-insulin analogs to the IGFR. Surprisingly, we have found that the affinity of SCI (His^{A8}, Asp^{B10}, Asp^{B28}, and Pro^{B29}) for the IGFR is similar to that of human insulin; any potential increase is <twofold. Since the Lys^{B28}-Pro^{B29} substitutions in Humalog confer a twofold increase in IGFR cross-binding without a detectable increase in risk of cancer in patients, the IGFR-binding properties of SCI (His^{A8}, Asp^{B10}, Asp^{B28}, and Pro^{B29}) (SEQ. ID. NO 36) are unlikely to be significant.

Based upon the foregoing disclosure, it should now be apparent that the single-chain insulin analogue provided herein will provide increased isoform-specific receptor binding relative to natural insulin with preferential binding to IR-A but without increased binding to IGFR. It is, therefore, to be understood that any variations evident fall within the scope of the claimed invention and thus, the selection of specific component elements can be determined without departing from the spirit of the invention herein disclosed and described.

1. A method of treating a mammal comprising administering a physiologically effective amount of an insulin analogue or a physiologically acceptable salt thereof where the insulin analogue displays more than twofold greater binding affinity to insulin receptor isoform A (IR-A) than insulin receptor isoform B (IR-B) and wherein the analogue has at least one third of the relative binding affinity to IR-B compared to wild type insulin from which the analogue is derived.

2. The method of claim 1, wherein the insulin analogue or a physiologically acceptable salt thereof displays a binding affinity for IR-A at least fourfold greater than for IR-B.

3. The method of claim 2, wherein the insulin analogue or a physiologically acceptable salt thereof is a single-chain insulin analogue or a physiologically acceptable salt thereof, containing an insulin A-chain sequence or an analogue thereof and an insulin B-chain sequence or an analogue thereof connected by a polypeptide of 4-13 amino acids.

4. The method of claim 3, wherein the polypeptide of 4-13 amino acids has the sequence Gly-Gly-Gly-Pro-Arg-Arg (SEQ. ID. NO. 19).

5. The method of claim 3, wherein the insulin analogue or a physiologically acceptable salt thereof is an analogue of a mammalian insulin.

6. The method of claim 5, wherein the insulin analogue or a physiologically acceptable salt thereof is an analogue of human insulin.

7. The method of claim 6, wherein the insulin analogue or a physiologically acceptable salt thereof is a polypeptide having a sequence selected from the group consisting of polypeptides having the sequence of SEQ. ID. NOS. 26 and 36.

8. An insulin analogue comprising a single-chain polypeptide, where the insulin analogue displays more than twofold greater binding affinity to insulin receptor isoform A (IR-A) than insulin receptor isoform B (IR-B) and where the insulin analogue has an affinity for Insulin-like Growth Factor Receptor no greater than that of natural insulin as measured in vitro.

9. The insulin analogue of claim 8, wherein the analogue displays selective binding to the A isoform of the insulin receptor by a factor of at least fourfold relative to binding the β isoform of the insulin receptor.

10. The insulin analogue of claim 9, comprising a polypeptide having a sequence selected from the group consisting of polypeptides having the sequence of SEQ. ID. NO. 17, wherein Xaa₄₋₁₃ is 6 of any amino acids, with the proviso that the first two amino acids of Xaa₄₋₁₃ are not arginine.

11. The insulin analogue of claim 9, comprising a single chain polypeptide of formula I,
B—C-A (I)
wherein B comprises a polypeptide having the sequence:
(SEQ. ID. NO. 38)
FVNQHLCGSX₂LVEALYLVCGERGFFYTX₃ X₄T
where X₂ is D or H, X₃ is P, D or K, and X₄ is K or P,
wherein C is a polypeptide consisting of the sequence GGGPRR (SEQ.ID. NO. 19), and
wherein A comprises a polypeptide having the sequence:

| | |
|---|---|
| GIVEQCCX₁SICSLYQLENYCN | (SEQ. ID. NO. 37) |

where X₁ is T or H.

12. The insulin analogue of claim 11, comprising a polypeptide selected from the group consisting of a polypeptide having the sequence of SEQ. ID. NO. 26 and a polypeptide having the sequence of SEQ. ID. NO. 36.

13. The insulin analogue of claim 12, comprising a polypeptide having the sequence of SEQ. ID. NO. 26.

14. The insulin analogue of claim 12, comprising a polypeptide having the sequence of SEQ. ID. NO. 36.

15. A nucleic acid encoding a single-chain insulin analogue according to claim 10.

16. An expression vector comprising the nucleic acid sequence of claim 15.

17. A host cell transformed with the expression vector of claim 16.

18. A nucleic acid encoding a single-chain insulin analogue according to claim 11.

19. A nucleic acid encoding a single-chain insulin analogue according to claim 12.

20. A nucleic acid encoding a single-chain insulin analogue according to claim 13.
